(19) United States
(12) Patent Application Publication
(10) Pub. No.: US 2006/0153931 A1
(43) Pub. Date: Jul. 13, 2006

(54) Prevention of flap necrosis in plastic surgery

(76) Inventors: Jonathan S Stamler, Chapel Hill, NC (US); Michael R Zenn, Chapel Hill, NC (US); Eric J Toone, Durham, NC (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/521,394

(22) PCT Filed: Dec. 2, 2002
(86) PCT No.: PCT/US02/36138

§ 371 Date: Aug. 5, 2005

(30) Foreign Application Priority Data
Dec. 6, 2001 (US) 60336175

(51) Int. Cl. A61K 31/724; A61K 31/21; A61K 33/24

(52) U.S. Cl. 424/617; 514/509; 514/58

(57) ABSTRACT

NO, NO donor or prodrug that causes formation of nitrosothiol in tissue optionally in combination with lidocaine is topically applied to pedicle of a pedicle flap or other source of blood supply to prevent necrosis in pedicle flap or in any microvascular surgery. Composition containing drug is formulated with nitrosylated polythiolated cyclodextrin to improve drug delivery.

### CROSS-REFERENCE TO RELATED APPLICATIONS

This application claims the benefit of U.S. Application No. 60/336,175, filed on Dec. 6, 2001, the whole of which is incorporated herein by reference.

### TECHNICAL FIELD

This invention is directed to a method for resolving or preventing vasoconstriction and preventing depletion of or restoring blood flow in a pedicle flap or any other microsurgery.

### BACKGROUND OF THE INVENTION

In plastic/microvascular surgery for providing new tissue where such is necessary because of traumatic or other injury or for reconstruction after surgery to remove cancerous and surrounding tissue, a flap may be used for reconstruction. Specifically, a flap is a block of tissue isolated on its nutrient blood supply. Any time tissue is transferred on its pedicle (artery and vein), it is subject to vasospasm and thrombosis which may lead to necrosis of the tissue, if uncorrected.

In an attempt to prevent flap necrosis, it is standard procedure to topically apply a vasodilator such as lidocaine or paparavine to pedicle which attaches the pedicle flap to its source of blood supply. This resolves or prevents vasoconstriction intraoperatively but that effect is not always effective for preventing depletion of or restoring blood flow. Moreover, this effect is short-lived, decreasing to about 50% or less of baseline 30 minutes after lidocaine application.

### SUMMARY OF THE INVENTION

It has been found herein that the effect of lidocaine in resolving or preventing vasoconstriction and preventing depletion of or restoring blood flow in a pedicle flap in plastic surgery or other microsurgery can be improved and lengthened by use of nitric oxide (NO) and/or NO donor and/or prodrug that causes formation of nitrosothiol in tissue as the only vasodilator or in combination with lidocaine compared to conventional use of lidocaine alone.

The invention herein in a first embodiment is directed to a method for preventing necrosis in a pedicle flap or in any microvascular surgery comprising topically applying to pedicle or other source of blood supply a therapeutically effective amount of vasodilator composition containing NO or NO donor or prodrug that causes formation of nitrosothiol in tissue optionally in combination with lidocaine.

The term “therapeutically effective amount” is used herein, in respect to the first embodiment, to mean a vasoconstriction resolving or preventing amount and blood flow depletion preventing or restoring amount.

The invention herein in a second embodiment is directed to a method for delivering a drug comprising incorporating the drug in a therapeutically effective amount in a gel or solution or in a pharmaceutical base containing from 1 μM to 100 mM nitrosylated polythiolated cyclodextrin or other nitrosylated polymer or long lived gel coating equivalent exemplified by cyclodextrin or coating the drug in pill form with coating that delivers nitric oxide and administering the resulting combination topically to the skin or topically to the gastrointestinal tract, thereby to improve the delivery of the drug by increasing absorption of the drug and/or to negate side effects of the drug and/or to obtain the combined effect of the drug and nitric oxide delivery.

### BRIEF DESCRIPTION OF THE DRAWING

FIG. 1 is a graph of time in minutes versus % baseline and sets for the results of Background Example 2.

### DETAILED DESCRIPTION

We turn now to the first embodiment of the invention, that is to the method for preventing necrosis in a pedicle flap or in any microvascular surgery comprising topically applying to the pedicle or other source of blood supply a therapeutically effective amount of vasodilation composition containing NO or NO donor or prodrug that causes formation of nitrosothiol in tissue optionally in combination with lidocaine.

We turn now to the NO, NO donors, and prodrugs that cause formation of nitrosothiol in tissue, that are topically applied in compositions in the first embodiment of the invention herein.

Nitric oxide is readily applied as a water solution.

We turn now to the NO donor which is administered. An NO donor donates nitric oxide or a related redox species and more generally provides nitric oxide bioactivity, that is activity which is identified with nitric oxide, e.g., vasorelaxation or stimulation or inhibition of a receptor protein, e.g., ras protein, adrenergic receptor, NFκB. NO donors including S-nitroso, O-nitroso, C-nitroso and N-nitroso compounds and nitro derivatives thereof and metal NO complexes, but not excluding other NO bioactivity generating compounds, useful herein, are described in “Methods in Nitric Oxide Research,” edited by Feelisch, M., and Stamler, J. S., John Wiley & Sons, New York, 1996, pages 71-115 which is incorporated herein by reference. NO donors which are C-nitroso compounds where nitroso is attached to a tertiary carbon which are useful herein include those described in U.S. patent application Ser. No. 09/695,934 which has matured into U.S. Pat. No. 6,359,182 and those described in WO 02/34705. Examples of S-nitroso compounds including S-nitrosothiols useful herein include, for example, S-nitrosoglutathione, S-nitroso-N-acetylpenicillamine, S-nitroso-cysteine and ethyl ester thereof S-nitroso cysteinyl glycine, S-nitroso-gamma-methyl-L-homocysteine, S-nitroso-L-homocysteine, S-nitroso-gamma-thio-L-leucine, S-nitroso-delta-thio-L-leucine, and S-nitrosoalbumin. An example of an S-nitrosylated or an O— and S-nitrosylated compound is nitrosylated polythiolated cyclodextrin (hereinafter cyclodextrin NO or CX—NO) as described in Stamler, et al U.S. Pat. No. 6,403,759 which can be, for example O— and S-nitrosylated β-cyclodextrin as described in Example 14 of U.S. Pat. No. 6,403,759 or nitrosylated perthiolated-β-cyclodextrin as described in Examples 3-6 of U.S. Pat. No. 6,403,759. Examples of other NO donors useful herein are metal nitrosyls such as sodium nitroprusside (nipride), alkyl nitrites of molecular weight up to 10,000 such as ethyl nitrite, nitroglycerin, SIN1 which is molsidomine, furoxamines, N-hydroxy(N-nitrosamine), perfluorocarbons that have been saturated with NO or a hydrophobic NO donor, and NO entrained in carbon monotubules.

Examples of C-nitroso compounds that are NO donors include:

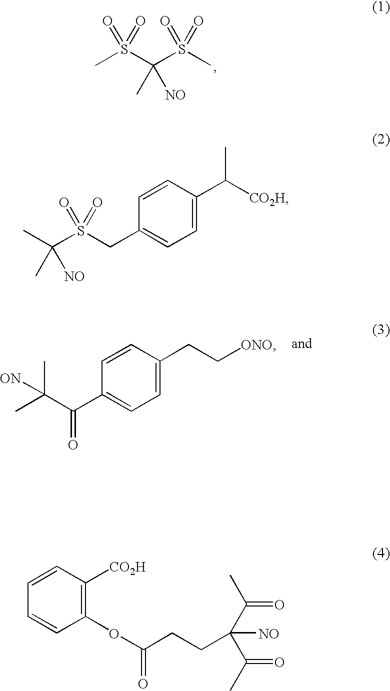

NO donors or NO prodrugs that causes formation of nitrosothiol in tissue, that are used in working examples are ethyl nitrite or nitrosylated polythiolated cyclodextrin.

As indicated above, the NO, NO donor and/or prodrug is administered in a therapeutically effective amount. In general, these are applied at a concentration ranging from 1 μM to 100 mM, with variation within the range depending on the agent administered. Ethyl nitrite is available as 90-95% ethyl nitrite in ethanol and can be applied as an ethanol solution at a concentration, e.g., of 5×10⁻³-5×10⁻⁴M. Cyclodextrin NO can be applied as a solution or gel or as fine particles in a pharmaceutical base at a concentration of 1 μM to 100 mM.

We turn now to where the NO, NO donor or prodrug that causes formation of nitrosothiol in tissue is used in combination with lidocaine. The lidocaine can be applied in solution or in a cream or as viscous lidocaine present in the applied composition, e.g., at 2 to 20%.

The NO, NO donor or prodrug that causes formation of nitrosothiol in tissue, and lidocaine, if used, can be topically applied in a liquid or viscous composition, e.g., as a solution, cream, ointment or gel.

We turn now to the method of the second embodiment herein which is directed to a method for delivering a drug (which is not nitrosylated) comprising incorporating the drug in a therapeutically effective amount in a gel or solution or in a pharmaceutical base containing from 1 μM to 100 mM nitrosylated polythiolated cyclodextrin (sometimes denoted CX—NO) or any nitrosylated polymer or long lived gel coating equivalent exemplified by cyclodextrin or coating the drug in pill form with a coating that delivers nitric oxide, e.g., a coating comprising CX—NO or other nitrosylated polymer or of nitric oxide entrained in carbon nanotubules and administering the resulting combination topically to the skin or topically to the gastrointestinal tract, thereby to improve the delivery of the drug and/or to negate side effects of the drug and/or to obtain the combined effect of the drug and nitric oxide delivery.

The nitrosylated polythiolated cyclodextrin is that described for the first embodiment.

The drugs can be, for example, aspirin or nonsteroidal anti-inflammatory drugs (NSAIDs) or selective inhibitors of cyclooxygenase-2, for treatment of pain or inflammation, e.g. for headache or osteoarthritis, or an antiproliferative agent, e.g. rapamycin or taxol, for the treatment of the kinds of cancer that the antiproliferative agent is therapy for, or proteins including hemoglobin as a blood substitute, factor VIII inhibitor for sepsis or insulin for the treatment of Type I diabetes. For example, CX—NO can be formulated with hemoglobin based blood substitutes at a ratio of hemoglobin to NO ranging from 25:1 to 1,000:1 to improve peripheral blood flow and mitigate hypertension.

The CX—NO can be formulated in a gel or solution or as fine particles in a pharmaceutical base and the drug later admixed or the drug and CX—NO can be formulated together in a gel or solution or pharmaceutical base or the drug can be incorporated before the CX—NO.

Composition containing the drug and CX—NO can be administered by application topically to the skin where it acts locally or can be incorporated in a capsule or other oral dosage form for application topically to the gastrointestinal tract or can be administered via inhalation for application to the gastrointestinal tract.

Composition constituted of component(s) comprising drug, in pill form, and coating on the pill that delivers NO, can be administered orally for application topically to the gastrointestinal tract.

The nitric oxide from the CX—NO increases absorption of the drug by causing vasodilation and/or increasing permeability and in such case is administered in a vasodilating and/or permeability increasing amount.

The method of the second embodiment can also alleviate side effects of a drug, e.g., the nitric oxide of the CX—NO will ameliorate or prevent gastrointestinal bleeding that can be caused by aspirin and NSAIDs.

The method of the second embodiment also can be an alternative to NO or SNO substitution of the drug as described in U.S. Pat. No. 6,057,367 and U.S. Pat. No. 6,359,182 to obtain the combined effect of drugs and NO or NO donor administration.

The invention is supported by the following background examples, which uses a standard model for determining flap blood flow. In the Background Example 1, ENO means ethyl nitrite. In the Background Example 2, CX—NO means nitrosylated polythiolated cyclodextin.

### BACKGROUND EXAMPLE 1

Male rats (N=9) were anesthetized and skin flaps (3×3 cm) were raised bilaterally based on the epigastric artery and vein. Pedicle vasoconstriction was induced by topically applying 10⁻⁵ M endothenin-1 (ET-1). Fifteen minutes after ET-1 application, either 2% lidocaine or 10⁻⁴M ENO, dissolved in saline, was applied. Over the subsequent 30 min, flap blood flow was measured with a laser Doppler flowmeter and the diameter of pedicle vessels was measured using videomicroscopy. Data are expressed as % of baseline (mean±SEM). Pairwise comparisons were performed using a Student's t-test. ET-1 caused a 69-76% vasoconstriction of the epigastric artery, 43-72% vasoconstriction of the epigastric vein, and a reduction of flap blood flow to 35-45% of baseline. Application of lidocaine caused a rapid arterial vasodilation to 101±6% of baseline within 1 min, but after 30 min the effect decreased to 82±6% of baseline. The vein dilated to 68±7% of baseline 30 min after lidocaine application. Lidocaine also caused an increase of blood flow to 87±13% of baseline in 2 min, but only 55±7% of baseline after 30 min. ENO slowly dilated the artery to 83±5% of baseline after 1 min and 98±5% of baseline after 30 min and dilated the vein to 75±5% of baseline after 30 min. ENO restored blood flow to 62±6% of baseline after 2 min and 86±10% of baseline after 30 min. ENO caused a greater restoration of blood flow and arterial diameter (p<0.05) compared to lidocaine beginning 13 min (for blood flow) or 10 min (for diameter) after application of each dilator. Lidocaine was shown to have a faster effect, but ENO had a longer lasting and greater effect on arterial dilation and blood flow.

### BACKGROUND EXAMPLE 2

Adult male rats (six) weighing 250-250 gm were anesthetized with intraperitoneal sodium pentobartital at initial doses of 50 mg/kg after being induced with isofluorane as an inhalational anesthetic. Supplemental pentobarbital was administered as needed. The rats' core temperature was measured via rectal probe and maintained at 36-38° C. with a heating pad. The groin and abdomen were shaved.

A tracheotomy was performed, and the rats intubated directly to help eliminate motion associated with respiratory movements. Bilateral 3×3 cm island skin flaps based upon the epigastic artery and vein were raised. The epigastic artery and vein were carefully isolated from each other and from surrounding soft tissue. Dissection was performed with the aid of a surgical microscope. After elevation of the flap, it was positioned on a clear acrylic sheet and maintained at its original dimensions with 4-0 nylon sutures. A laser Doppler flow probe was placed on the center of the flap. A video camera was connected to the camera port opening of the microscope. Camera output was connected to a video timer, a 13-inch video monitor, and a videotape recorder. Diameter measurements were made using a digital caliper with 100-micrometer resolution. After dissection, the rats were allowed to stabilize for 60 minutes before baseline measurements were recorded and the experiment begun in order to better help control any vascular changes associated with the preparation of the model.

Vasoconstriction was induced by adding a drop of Endothelin-1 (ET-1) at 10⁻⁵ M concentration directly to the exposed vascular pedicle using a 27 gauge needle on a tuberculin syringe. ET-1 was dissolved in 0.1% acetic acid and stored at 20° C. Data collected each subsequent minute included the arterial diameter, vein diameter, and laser Doppler flow. Fifteen (15) minutes following the administration of ET-1, 0.25 ml of 3.17 mM CX—NO in dimethylsulfoxide (DMSO) was applied. Data were gathered for an additional thirty (30) minutes. The rats were then sacrificed with an overdose of pentobarbital.

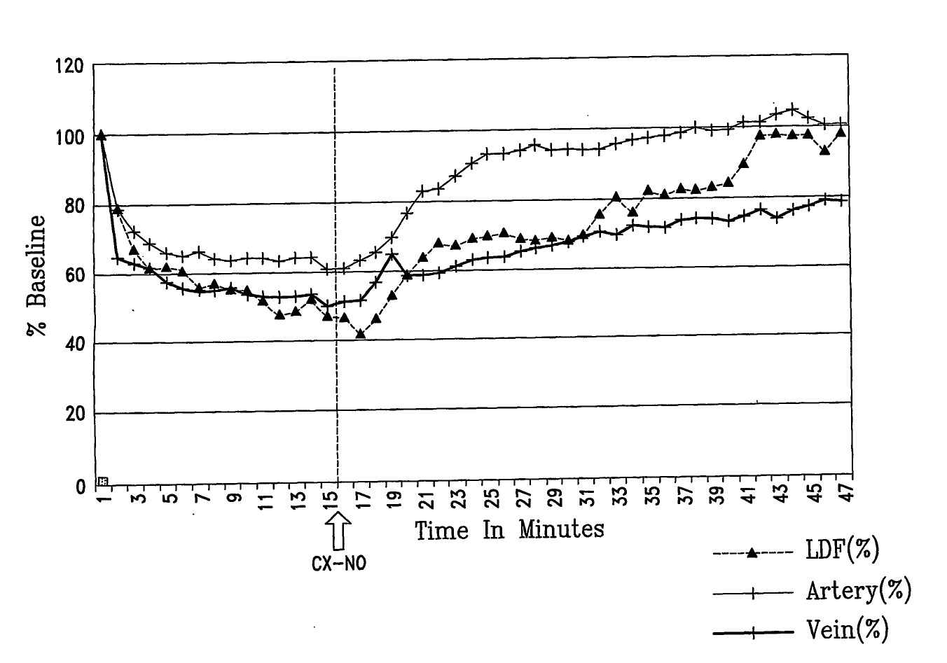

The results are shown in FIG. 1 where the upper curve is for arterial diameter as a percentage of baseline, the lowest curve is for vein diameter as a percentage of baseline and the dashed curve shows measurement of blood flow with a laser Doppler flowmeter and shows blood flow in small vessels as a percentage of baseline flow. The results show that the CX—NO caused increase in arterial and vein diameter and increase in blood flow in small vessels.

The invention is illustrated in the following working examples.

### EXAMPLE I

A 40-year-old white male with a head and neck tumor resected and reconstructed with a free tissue transfer of forearm tissues to the face develops vasospasm after reattachement and no blood flow is seen. Topical application of ethyl nitrite (100 μM) results in vasodilation and flow is restored. The same result is obtained when compound (1) described above is applied at a concentration of 50 mM instead of the ethyl nitrite. The same result is obtained when sodium nitroprusside is applied at a concentration of 50 mM instead of the ethyl nitrite.

### EXAMPLE II

A 60-year-old undergoing emergent CABF (coronary artery bypass) develops cardiac ischemia after grafting, from vasospasm of the grafts. Topical application of lidocaine (2%) resulted in temporary but marginal blood flow. Addition of 100 μM ethyl nitrite restored flow and normalized EKG (ischemia).

### EXAMPLE III

CX—NO of Example 14 of U.S. Pat. No. 6,403,759 is formulated in a gel at 50 mM and is applied topically to skin to relieve pain of osteoarthritis.

### EXAMPLE IV

CX—NO of Example 14 of U.S. Pat. No. 6,403,759 is formulated in a gel at 50 mM and is admixed with aspirin and the combination used to fill capsules each containing 100 mg aspirin and 200 mg of CX—NO. Administration of capsules orally relieved headache without any gastrointestinal bleeding. Alternatively, the CX—NO is used as or included in a coating for aspirin tablets (100 mg aspirin in a tablet), and administration orally of the coated aspirin provides the same result.

### EXAMPLE V

The CX—NO of Example 14 of U.S. Pat. No. 6,403,759 is formulated in a gel at 50 mM together with a therapeutic amount of insulin. Application to skin is a treatment for Type I diabetes.

### EXAMPLE VI

The CX—NO of Example 14 of U.S. Pat. No. 6,403,759 is formulated with hemoglobin based blood substitute in a ratio of hemoglobin to NO of 500:1. The formulation is administered to a patient to improve peripheral blood flow and mitigate hypertension.

Variations

Variations will be obvious to those skilled in the art. Thus, the scope of the invention is defined by the claims.

What is claimed is:

1. A method for preventing necrosis in a pedicle flap or in any microvascular surgery comprising topically applying to pedicle or other source of blood supply, a therapeutically effective amount of vasodilator composition containing NO or NO donor or prodrug that causes formation of nitrosothiol in tissue, optionally in combination with lidocaine.

2. The method of claim 1 where the vasodilator composition contains alkyl nitrite of molecular weight up to 10,000.

3. The method of claim 2 where the alkyl nitrite is ethyl nitrite.

4. The method of claim 1 where the vasodilator composition contains an S— nitrosothiol.

5. The method of claim 4 where the S-nitrosothiol is cyclodextrin NO.

6. The method of claim 1 where the vasodilator composition contains a metal nitrosyl.

7. A method for delivering a drug comprising incorporating the drug in a gel or in a solution or in a pharmaceutical base, containing from 1 μM to 100 μM nitrosylated polythiolated cyclodextrin or other nitrosylated polymer or long lived gel coating equivalent exemplified by cyclodextrin or coating the drug in pill form with a coating that delivers nitric oxide, and administering the resulting drug containing composition topically to the skin or topically to the gastrointestinal tract.

8. The method of claim 7 where the drug is incorporated in a therapeutically effective amount in a gel containing from 1 μM to 100 mM nitrosylated polythiolated cyclodextrin.
